(19) United States
(12) Patent Application Publication
(10) Pub. No.: US 2020/0393407 A1
(43) Pub. Date: Dec. 17, 2020

(54) CROSS-GAP-NANOPORE HETEROSTRUCTURE DEVICE AND METHOD FOR IDENTIFYING CHEMICAL SUBSTANCE

(71) Applicant: University of Kentucky Research Foundation, Lexington, KY (US)

(72) Inventor: Douglas R. Strachan, Lexington, KY (US)

(21) Appl. No.: 16/901,393

(22) Filed: Jun. 15, 2020

Related U.S. Application Data
(60) Provisional application No. 62/861,090, filed on Jun. 13, 2019.
(60) Provisional application No. 63/037,689, filed on Jun. 11, 2020.

(51) Int. Cl. G01N 27/414; G01N 33/487; C12Q 1/6816

(52) U.S. Cl. CPC G01N 27/4146; B82Y 15/00; C12Q 1/6816; G01N 33/48721

(57) ABSTRACT

A heterostructure device and method allow for detection and identification of a chemical substance. The device includes one or more atomically-thin conducting layers and one or more atomically-thin insulating layers including one or more nanogaps that cross and form one or more nanopores.

### RELATED APPLICATION

This application claims priority to U.S. Provisional Patent Application Ser. No. 62/861,090, filed on Jun. 13, 2019, and U.S. Provisional Patent Application Ser. No. 63/037,689, filed on Jun. 11, 2020, the full disclosures of which are incorporated herein by reference.

### GOVERNMENT SUPPORT

This invention was made with government support under Grant No. 1603152 awarded by the National Science Foundation (NSF) through Chemical, Bioengineering, Environmental, and Transport Systems (CBET). The government has certain rights in the invention.

### TECHNICAL FIELD

This document relates generally to devices and methods for determining a chemical substance and, more particularly, to cross-gap-nanopore devices and related methods for determining a chemical substance such as the nucleotide sequence of a strand of ribonucleic acid (RNA) or deoxyribonucleic acid (DNA).

### BACKGROUND

Nanopores consisting of integrated electrodes at the location of the pore could be of tremendous technological use in filtering biological and chemical substances from solutions and in sensing and sequencing molecules, like DNA and RNA, as they translocate through the pore. Such sequencing would have use in the rapid sequencing for human genome and infectious diseases with small test samples. Due to the relatively short timescales for mutations of infections, such as RNA viruses, being able to quickly and efficiently track their genetic variations and progressions within society is extremely important for treating and understanding such diseases.

However, to achieve such capabilities has been hindered due to the fact that it is extremely difficult to accurately and reliably determine genetic sequencing with high throughput using minimal amounts of targeted genetic molecules (i.e., DNA or RNA). High throughput methods in real-time with electrical detection through nanopore translocation could enable this accurate and reliable genetic sequencing of individual genetic molecules. However, current technologies are not yet capable of obtaining the required precise placement of multiple electrodes, having sufficient electrical isolation from each other, at the location of a nanopore.

Advantageously, the cross-gap-nanopore heterostructure device disclosed herein allows precise placement of multiple electrodes at the location of a nanopore while having sufficient electrical isolation from each other to function in an efficient and effective manner.

### SUMMARY

In accordance with the purposes and benefits described herein, a new and improved heterostructure device is provided for identifying or determining a chemical substance including polymers, proteins, genetic material such as strands of DNA and RNA, individual molecules, particles and the like. That heterostructure device comprises; (a) a first conducting layer including a first nanogap, (b) a first insulating layer including a second nanogap and (c) a first nanopore formed at a first crossing point of the first nanogap and the second nanogap. The first nanopore extends through the first conducting layer and the first insulating layer. In one or more particularly useful embodiments, the first nanogap forms a first electrode pair.

In one or more embodiments of the heterostructure device, the first conducting layer is atomically-thin. In one or more embodiments of the heterostructure device, the first insulating layer is atomically-thin. In one or more embodiments of the heterostructure device, both the first conducting layer and the first insulating layer are atomically-thin.

For purposes of this document, the terminology “atomically-thin” means having a thickness of about 1 nm or less.

In one or more possible embodiments of the heterostructure, the heterostructure includes a second atomically-thin conducting layer wherein the first atomically-thin insulating layer is sandwiched between the first atomically-thin conducting layer and the second atomically-thin conducting layer. The second atomically-thin conducting layer includes a third nanogap that crosses the first nanogap and the second nanogap at the first crossing point so that the first nanopore also extends through the second atomically-thin conducting layer.

In one or more possible embodiments of the heterostructure, the first nanogap forms a first electrode pair within the first nanopore and the second nanogap forms a second electrode pair within the first nanopore.

In one or more possible embodiments of the heterostructure, the first conducting layer and the second conducting layer include additional nanogaps that cross the second nanogap in the atomically-thin insulating layer at a second crossing point forming a second nanopore.

In one or more possible embodiments of the heterostructure, additional nanogaps in the first conducting layer and the second conducting layer form additional electrode pairs within the second nanopore.

In one or more possible embodiments of the heterostructure, the heterostructure further includes alternating additional atomically-thin conducting layers and additional atomically-thin insulating layers providing (a) additional electrode pairs in the first nanopore and the second nanopore, (b) additional nanopores at additional crossing points or (c) additional electrode pairs in the first nanopore and the second nanopore and additional nanopores at additional crossing points.

In one or more possible embodiments of the heterostructure, the first atomically-thin conducting layer, the second atomically-thin conducting layer and the additional atomically-thin conducting layers are made from a material selected from a group consisting of graphene, transition metal dichalcogenides (TMDs), borophene, germanene, silicene, stanene, plumbene, phosphorene, antimonene, Si₂BN, borocarbonitrides and combinations thereof.

In one or more possible embodiments of the heterostructure, the first atomically-thin insulating layer and the additional atomically-thin insulating layers are made from a material selected from a group consisting of hexagonal boron nitride, transition metal dichalcogenides (TMDs), bismuthene, borocarbonitrides and combinations thereof.

In accordance with yet another aspect, a cross-gap-nanopore heterostructure is provided. That cross-gap-nanopore heterostructure is adapted for the real-time determination of nucleotide sequencing of a strand of genetic material. In one or more of the many possible embodiments, that genetic material comprises RNA. In one or more of the many possible embodiments, that genetic material comprises DNA. In one or more of the many possible embodiments, that genetic material is a combination of RNA and DNA.

In one or more of the many possible embodiments of the cross-gap-nanopore heterostructure, the cross-gap-nanopore heterostructure may include (a) a plurality of alternating atomically-thin conducting layers and insulating layers and (b) at least one nanopore having stacked electrode pairs. In one or more of the many possible embodiments of the cross-gap-nanopore heterostructure, the cross-gap-nanopore heterostructure may include (a) a plurality of alternating atomically-thin conducting layers and insulating layers and (b) a plurality of nanopores wherein each nanopore of the plurality of nanopores includes at least one individually addressable electrode pair. The cross-gap-nanopore structure may be supported on any suitable porous substrate.

In accordance with yet another aspect, a method of determining a chemical species is also provided. That method includes the step of passing the chemical species through at least one nanopore in a cross-gap-nanopore heterostructure.

The method may also include additional steps including, but not necessarily limited to:

(a) performing lateral electrical detection of the chemical substance as the chemical substance passes through the at least one nanopore;

(b) applying a voltage difference to a first electrode pair in the at least one nanopore;

(c) applying voltage differences to a plurality of different electrode pairs in the at least one nanopore;

(d) simultaneously electrically probing the chemical substance with the plurality of different electrode pairs in the at least one nanopore as the chemical substance passes through the at least one nanopore;

(e) influencing the flow of the chemical substance through the at least one nanopore through application of dielectrophoretic forces; and

(f) combinations of (a)-(e).

In the following description, there are shown and described several embodiments of the new and improved (a) heterostructure devices, (b) cross-gap-nanopore heterostructures, and (c) methods of determining a chemical substance. As it should be realized, the heterostructure devices, cross-gap-nanopore heterostructures and methods are capable of other, different embodiments and their several details are capable of modification in various, obvious aspects all without departing from the heterostructures devices and methods as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the new and improved heterostructure devices, cross-gap-nanopore heterostructures and related methods for determining a sequence of a nucleotide strand and together with the description serve to explain certain principles thereof.

FIG. 1 is a schematic view illustrating one atomically-thin etched insulating layer and one atomically-thin etched conducting layer used to form a single cross-gap-nanopore heterostructure, of which an integrated electrode pair could embody one of the two etched materials.

FIG. 2 illustrates the layers of FIG. 1 stacked together resulting in an individual nanopore of which could have an electrode pair.

FIG. 3 a detailed schematic cross sectional view of a strand of nucleic acid passing through the nanopore of FIG. 2.

FIG. 4 schematically illustrates the heterostructure device of FIG. 3 supported over a pore in a support substrate with electrical contacts of the electrode pair connected to a voltage source and an ammeter.

FIG. 5 schematically illustrates three atomically-thin etched conducting and insulating layers used to form an integrated electrode cross-gap-nanopore heterostructure.

FIG. 6 illustrates the layers of FIG. 5 stacked together resulting in a series of individual nanopores each having a unique combination of electrodes.

FIG. 7 is a detailed schematic top plan view of one nanopore of the plurality of nanopores illustrated in FIG. 6.

FIG. 8 is a detailed schematic cross sectional view of a cross-gap-hetero structure including multiple nanopores.

FIG. 9 illustrates yet another possible embodiment of the cross-gap-nanostructure device including a larger number of alternating atomically-thin etched conducting layers and atomically-thin etched insulating layers.

Reference will now be made in detail to the present preferred embodiments of the cross-gap-nanopore heterostructure devices, heterostructures and related methods, examples of which are illustrated in the accompanying drawing figures.

### DETAILED DESCRIPTION

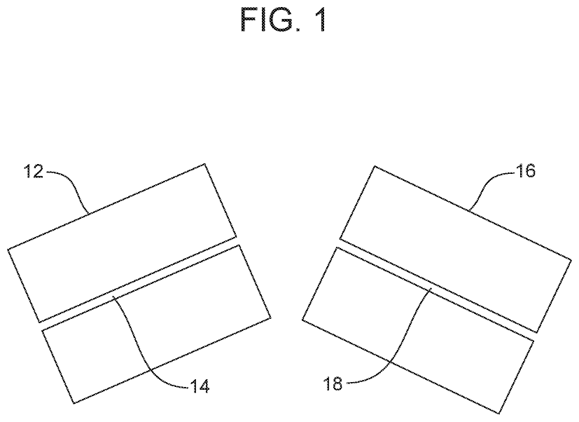

Reference is now made to FIGS. 1-3 which illustrate a first embodiment of a cross-gap-nanopore heterostructure device 10. That device 10 includes (a) a first atomically-thin conducting layer 12 having a first nanogap 14 and (b) a first atomically-thin insulating layer 16 having a second nanogap 18.

The first atomically-thin conducting layer 12 may be made from any appropriate material suitable for use as an atomically-thin conducting layer including, but not necessarily limited to graphene, transition metal dichalcogenides (TMDs), borophene, germanene, silicene, stanene, plumbene, phosphorene, antimonene, Si₂BN, borocarbonitrides and combinations thereof. The conducting layer 12 may have a thickness of about 1 nm or less.

The first atomically-thin insulating layer 16 may be made from any appropriate material suitable for use as an atomically-thin insulating layer including, but not necessarily limited to hexagonal boron nitride, transition metal dichalcogenides (TMDs), bismuthene, borocarbonitrides and combinations thereof. The insulating layer 16 may have a thickness of about 0.3 nm to 5 nm. For certain applications, either or both the conducting layer 12 and the insulating layer 16 may be much thicker and even have a thickness up to 100 nm or even a micron. Where both the conducting layer(s) 12 and the insulating layer(s) 16 are atomically thin, it is possible to detect individual nucleotides in a DNA or RNA molecule. Here it should also be appreciated that the cross-gap-nanopore heterostructures may comprise substantially any layered combination of conducting, semiconducting and insulating layers with the same or varying thicknesses from atomically thin up to and including one micron.

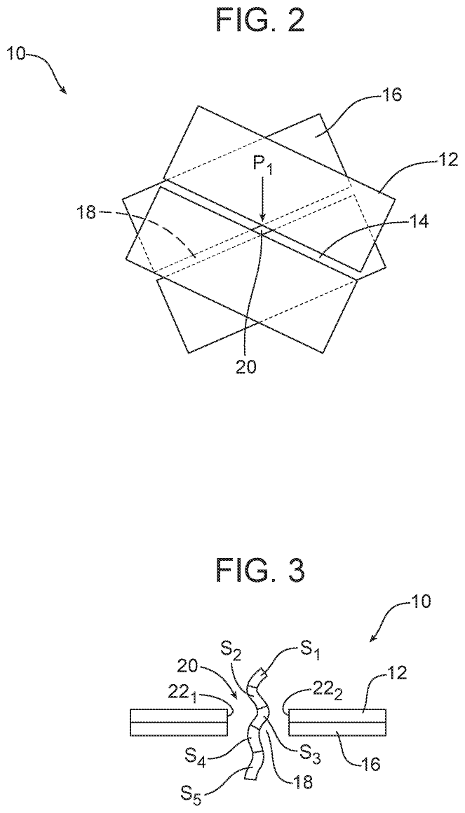

As illustrated in FIG. 2, when the first atomically-thin conducting layer 12 and first atomically-thin insulating layer 16 are stacked together a first nanopore 20 is formed at a first crossing point P₁ of the first nanogap 14 and the second nanogap 18 (i.e. the nanogaps cross at a non-zero angle). As should be appreciated, the first nanopore 20 extends through both of the layers 12, 16. The nanogaps 14, 18 are achieved by one-dimensional etching completely through the two dimensional layers 12, 16. The etch tracks or nanogaps 14, 18 may be very narrow (on the order of 10 nm or less) in width and may have a length greater than 100 nm and may be formed in parallel resulting in arrays. One possible approach for completing the etching of the nanogaps in the conducting layer 12 and the insulating layer 16 is disclosed in US 2020/0071817, the full disclosure of which is incorporated herein by reference.

As illustrated in FIG. 3, two of the four edges are formed by physically separated conducting sheets and the first nanogap 14 may form a first electrode pair 22₁, 22₂. That electrode pair 22₁, 22₂ may be used to perform lateral electrical detection of molecules and particles/chemical substance CS passing through the nanopore 20. In the illustrated embodiment, the chemical substance CS is a complex chemical substance. Such a complex chemical substance CS may include, for example, (a) deoxyribonucleic acid (DNA) or ribonucleic acid (RNA) wherein the segments S₁-S₅ of the chemical substance represent different nucleic acids or (b) a protein wherein the segments S₁-S₅ represent different amino acids. Of course, it should be appreciated that the chemical substance CS to be detected or identified could be substantially any molecule, particle, ion, or element.

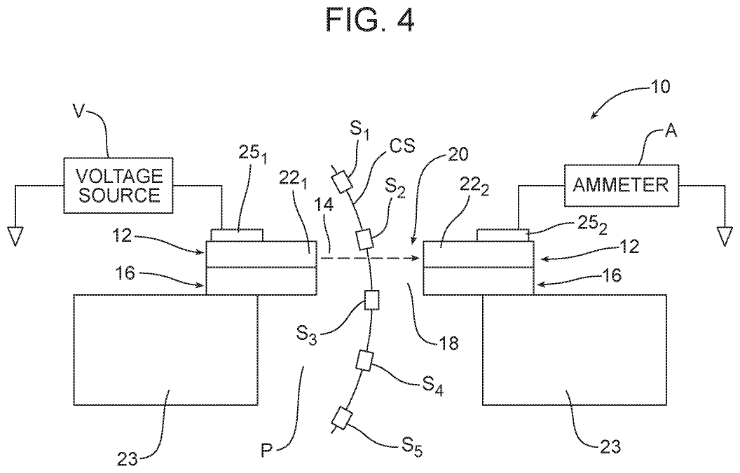

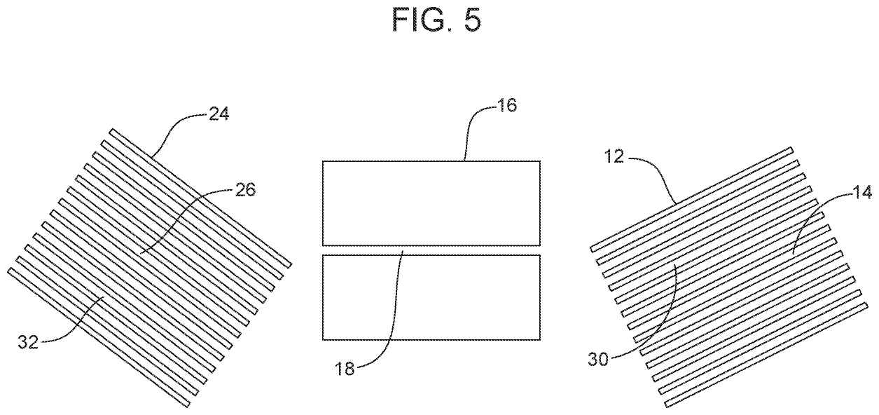

More particularly, as illustrated in FIG. 4, the heterostructure device 10 of FIG. 3 may be supported on a pore P in a porous support substrate or free-standing membrane 23 that may be made from any number of appropriate materials including, but not necessarily limited to, silicon, silicon dioxide (SᵢO₂), silicon nitride (SᵢNₓ), or the like.

Standard nano and micron lithography methods are then used to make electrical contacts 25₁, 25₂ to the two electrodes 22₁, 22₂ of the first conducting layer 12. The electrical contact 25₁ to the electrode 22₁ is connected to a voltage source V while the electrical contact 25₂ to the second electrode 22₂ is connected to a current (ammeter) meter A.

In operation, the voltage source V induces a sensing current (denoted by the dashed line across the nanopore 20) that passes through the molecule/chemical substance CS which is measured by the external ammeter A and distinguishes between the various building blocks of the molecules as it translocates through the nanopore 20.

Cross-gap-nanostructure devices 10 may be made with multiple conducting layers and/or multiple insulating layers that are stacked in an alternating pattern. Since alignment of etch tracks/nanogaps can be technically challenging, the conducting layers and/or insulating layers may be made with multiple etch tracks/nanogaps that increase the probability of aligning etch tracks/nanogaps in multiple stacked layers of conducting and insulating materials while also providing the possibility of the construction of an array of independently electronically addressable nanopores.

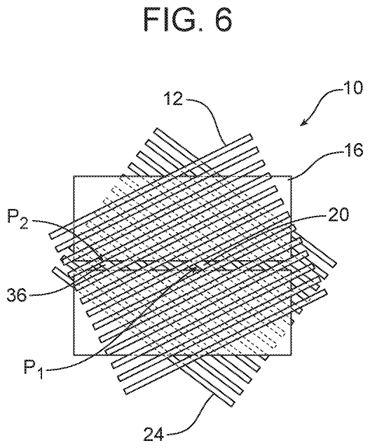

Reference is now made to FIGS. 5-8 illustrating a second possible embodiment of cross-gap-nanopore heterostructure device 10 including (a) a first atomically-thin conducting layer 12 having a plurality of nanogaps including a first nanogap 14, (b) a first atomically-thin insulating layer 16 having a second nanogap 18 and (c) a second atomically-thin conducting layer 24 having a plurality of nanogaps including a third nanogap 26. When the first atomically-thin conducting layer 12, first atomically-thin insulating layer 16 and second atomically-thin conducting layer 24 are stacked in alternating fashion, the first atomically-thin insulating layer 16 is sandwiched between the two atomically-thin conducting layers 12, 24 (see FIGS. 6 and 8).

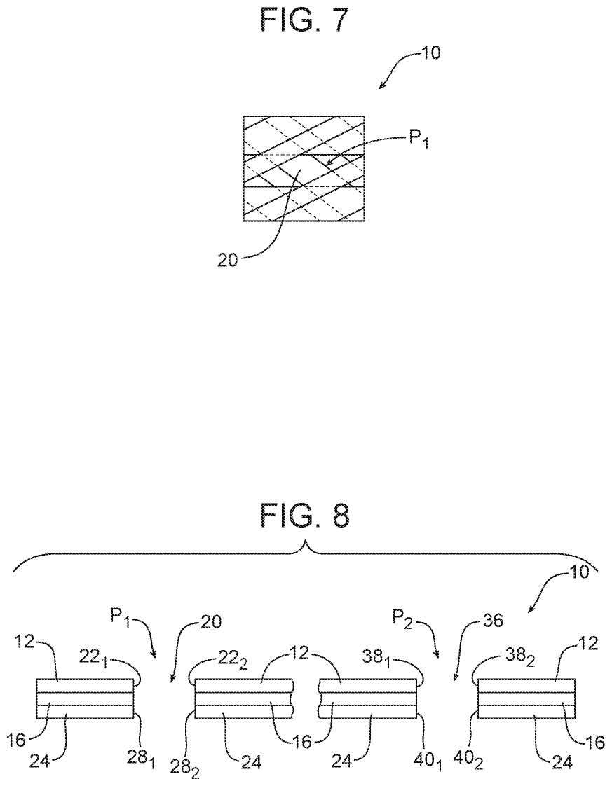

As shown in FIGS. 7 and 8, the third nanogap 26 crosses the first nanogap 14 and the second nanogap 18 at the first crossing point P₁ so that the first nanopore 20 also extends through the second atomically-thin conducting layer 24. The third nanogap 26 may form a second electrode pair 28₁, 28₂ within the first nanogap 20.

As illustrated in FIGS. 5 and 6, the first conducting layer 12 and the second conducting layer 24 may include additional nanogaps 30, 32, respectively, that cross the second nanogap 18 in the insulating layer 16 at a second crossing point P₂ forming a second nanopore 36.

The nanogap 30 in the first conducting layer 12 may form a third electrode pair 38₁, 38₂ within the second nanopore 36 while the second conducting layer may form a fourth electrode pair 40₁, 40₂ within the second nanopore.

The side view slice of the structure (FIG. 8) shows that we now have four electrically isolated electrodes 22₁, 22₂, 28₁, 28₂ that form the sidewalls of the nanopore 20 and four electrically isolated electrodes (38₁, 38₂) and (40₁, 40₂) that form the sidewalls of the nanopore 36. These four electrodes (22₁, 22₂), (28₁, 28₂) and (38₁, 38₂), (40₁, 40₂) allow for the ability to simultaneously control the voltage drops along the length of the nanopores 20, 36 and transversely to it, thus providing greater electrical control of its porosity. In addition, the two pairs of electrodes (22₁, 22₂), (28₁, 28₂) and (38₁, 38₂), (40₁, 40₂) now have the ability to simultaneously probe the electrical response of the pore contents at its bottom and top surfaces. This simultaneous electrical probing can permit time of flight detection of species as they move through the pore, while also permitting cross checking of measurements which is especially important for complex molecular detection (as in DNA sequencing).

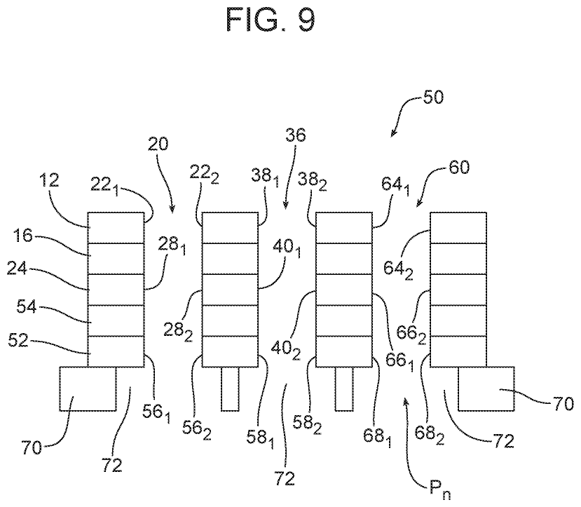

As illustrated in FIG. 9, the cross-gap-nanopore heterostructure device 50 may include any number of alternating additional atomically-thin conducting layers 52 and additional atomically-thin insulating layers 54 providing (a) additional electrode pairs 56₁, 56₂ in the first nanopore 20 and additional electrode pairs 58₁, 58₂ in the second nanopore 36, (b) additional nanopores 60 at additional crossing points Pₙ or (c) both additional electrode pairs in first and second nanopores and additional nanopores at additional crossing points. Those additional nanopores 60 may, in turn, include one or more additional electrode pairs (64₁, 64₂) in the first atomically-thin conducting layer 12, (66₁, 66₂) in the second atomically-thin conducting layer 24 and (68₁, 68₂) in the additional atomically-thin conducting layer 52.

As also illustrated in FIG. 9, the cross-gap-nanopore heterostructure device 50 may be stacked upon and supported by a porous support substrate 70 including a plurality of pores 72. Those pores 72 may be larger than the nanopores 20, 36, 60 and are typically microns or more. Where the pores 72 align with the nanopores 20, 36, 60, chemical substances may flow through the heterostructure device 50 and the porous support substrate 70.

The porous support substrate 66 may be made from any appropriate material including, but not necessarily limited to SiNₓ membrane frame, SiO₂ and Si. Further, while not shown in FIGS. 1-8, the heterostructure devices 10, 10′ could also be stacked or supported upon such a substrate.

In all cases, the various etched 2D layers are stacked through various mechanical transfer schemes that have been developed (using, e.g., sacrificial polymer layers that maintain the etch track dimensions and structure). This stacking onto the substrate holes can be achieved either after the holes in the substrate are formed, or prior to their formation utilizing a selective etch that does not disturb the 2D multilayer pore structure of interest. Moreover, it is also possible that these structures can be obtained through 1D etching procedures performed after their placement (or growth) on a substrate.

The cross-gap-nanopore heterostructure devices 10, 50 are useful in a method of determining a chemical substance CS. That method may be broadly described as including the step of passing the chemical substance CS through at least one nanopore 20, 36, 60 in a cross-gap-nanopore heterostructure 10, 50.

The passage of the chemical substance CS through the nanopore 20 (aka the translocation process) can be achieved in several ways as listed below:

1. Let the molecules diffuse through the nanopore. This is essentially a random process, using naturally occurring random fluctuations in the environment to push the molecules through.

2. Pumping a solution through the membrane to drive the molecules through.

3. Using surface energy gradients to drive the molecules through the membrane. By placing a drop of liquid with the molecules on one side of the membrane, the tendency for the drop to spread to the other side of the nanopore will drive molecules through the pore.

4. Using a density gradient of molecules across the nanopore will tend to yield the translocation of molecules from the higher density side to the lower density side.

5. Using “external electrodes” to drive ions contained in solution through the nanopore that will tend to subsequently drive the molecule of interest through the pore.

6. Using “external electrodes” to directly drive the molecule of interest (the one to be detected) through the nanopore. The driving mechanism can be through the molecule being charged or through its polarizability (i.e., a dielectrophoretic effect).

7. The integrated electrode pairs to the cross-gap nanopores can have a constant or alternating voltage applied between them that will tend to pull molecules of interest into the nanopore.

The method may include performing lateral electrical detection of the chemical substance CS as the chemical substance passes through the at least one nanopore 20, 36, 60. This is accomplished by applying a voltage difference to a first electrode pair 22₁, 22₂ in the at least one nanopore 20. In some embodiments, the method includes applying voltage differences to a plurality of different electrode pairs (22₁, 22₂), (28₁, 28₂), (38₁, 38₂), (40₁, 40₂), (64₁, 64₂), (66₁, 66₂) and (68₁, 68₂) in at least one nanopore 20, 36, 60.

The method may include the step of simultaneously electrically probing the chemical substance CS with the plurality of different electrode pairs (22₁, 22₂), (28₁, 28₂), (38₁, 38₂), (40₁, 40₂), (64₁, 64₂), (66₁, 66₂) and (68₁, 68₂) in the at least one nanopore 20, 36, 60 as the chemical substance passes through the at least one nanopore.

Still further, the method may include the step of influencing the flow of the chemical substance CS through the at least one nanopore 20, 36, 60 by application of dielectrophoretic forces.

Numerous benefits and advantages are provided by the cross-gap-nanopore heterostructure devices 10, 50 disclosed herein. The provision of multiple electrode pairs within a single nanopore allow for the ability to simultaneously control the voltage drops along the length of the nanopore and transversely to it—thus providing greater electrical control of its porosity. In addition, the multiple pairs of electrodes now have the ability to simultaneously probe the electrical response of the pore contents at its bottom and top surfaces as well as multiple points in between. This allows for the transverse simultaneous current detection of different portions of the same molecule as it translocates through the pore. This simultaneous electrical probing can permit time of flight detection of species as they move through the pore, while also permitting cross checking of measurements—especially important for complex molecular detection (as in DNA and RNA sequencing).

One of the important advantages of the multiple electrode pair formulation of cross-gapped nanopores is that it permits much greater accuracy in the sequencing determination. First, the second pair allows for a greater number of measurements of the same molecule which helps to significantly reduce read errors. Second, this configuration allows for the determination of changes of molecular directional switches to allow for the correct real-time sequencing. Using only one of the electrode pairs presents challenges in detecting the correct sequence. With the second electrode pair measurements, the change from leading to lagging gives unambiguous real-time determination of the translocation directional change that also allows for correct sequencing. By using additional electrode stacks, even greater improvements to the accuracy of the sequencing measurements can be achieved.

The foregoing has been presented for purposes of illustration and description. It is not intended to be exhaustive or to limit the embodiments to the precise form disclosed. Obvious modifications and variations are possible in light of the above teachings. For example, while the insulating layers 16, 54 in the above described embodiments include only a single etch track/nanogap 18, it should be appreciated that the insulating layers may also include an array of etch tracks/nanogaps to increase the probability of nanopore formation by alignment of those etch tracks/nanogaps with etch tracks/nanogaps of the other conducting and insulating layers of the heterostructure.

The cross-gap-nanopore heterostructures may also consist of the following alternative combinations. The cross-gap-nanopore heterostructures may consist only of conducting layers, only of insulating layers and only of semiconducting layers. The cross-gap-nanopore heterostructures may also not permit electrode pairs. Such novel nanopores could still provide detection and sequencing of molecular species having different properties and dimensions.

All such modifications and variations are within the scope of the appended claims when interpreted in accordance with the breadth to which they are fairly, legally and equitably entitled.

What is claimed:

1. A heterostructure device, comprising:
a first conducting layer including a first nanogap;
a first insulating layer including a second nanogap; and
a first nanopore formed at a first crossing point of the first nanogap and the second nanogap wherein the nanopore extends through the first conducting layer and the first insulating layer.

2. The heterostructure of claim 1, wherein the first conducting layer is atomically thin.

3. The heterostructure of claim 2, wherein the first insulating layer is atomically thin.

4. The heterostructure of claim 3, wherein the first nanogap forms a first electrode pair.

5. The heterostructure of claim 3, further including a second atomically-thin conducting layer including a third nanogap, wherein (a) the first atomically-thin insulating layer is sandwiched between the first atomically-thin conducting layer and the second atomically-thin conducting layer and (b) the third nanogap crosses the first nanogap and the second nanogap at the first crossing point so that the first nanopore also extends through the second atomically-thin conducting layer.

6. The heterostructure of claim 5, wherein the first nanogap forms a first electrode pair within the first nanopore and the second nanogap forms a second electrode pair within the first nanopore.

7. The heterostructure of claim 6, wherein the first conducting layer and the second conducting layer include additional nanogaps that cross the second nanogap in the atomically-thin insulating layer at a second crossing point forming a second nanopore.

8. The heterostructure of claim 7, wherein the additional nanogaps in the first conducting layer and the second conducting layer form additional electrode pairs within the second nanopore.

9. The heterostructure of claim 7 further including alternating additional atomically-thin conducting layers and additional atomically-thin insulating layers providing (a) additional electrode pairs in the first nanopore and the second nanopore, (b) additional nanopores at additional crossing points or (c) additional electrode pairs in the first nanopore and the second nanopore and additional nanopores at additional crossing points.

10. The heterostructure of claim 9, wherein the first atomically-thin conducting layer, the second atomically-thin conducting layer and the additional atomically-thin conducting layers are made from a material selected from a group consisting of graphene, transition metal dichalcogenides (TMDs), borophene, germanene, silicene, stanene, plumbene, phosphorene, antimonene, Si₂BN, borocarbonitrides and combinations thereof.

11. The heterostructure of claim 10, wherein the first atomically-thin insulating layer and the additional atomically-thin insulating layers are made from a material selected from a group consisting of hexagonal boron nitride, transition metal dichalcogenides (TMDs), bismuthene, borocarbonitrides and combinations thereof.

12. A cross-gap-nanopore heterostructure adapted for real-time determination of nucleotide sequencing of a strand of genetic material.

13. The cross-gap-nanopore heterostructure of claim 12, wherein the genetic material is selected from a group consisting of RNA, DNA and combinations thereof.

14. The cross-gap-nanopore heterostructure of claim 12, including (a) a plurality of alternating atomically-thin conducting layers and insulating layers and (b) at least one nanopore having stacked electrode pairs.

15. The cross-gap-nanopore heterostructure of claim 12, including (a) a plurality of alternating atomically-thin conducting layers and insulating layers and (b) a plurality of nanopores having at least one individually addressable electrode pair.

16. A method of determining a chemical substance, comprising:
passing the chemical substance through at least one nanopore in a cross-gap-nanopore hetero structure.

17. The method of claim 16, including performing lateral electrical detection of the chemical substance as the chemical substance passes through the at least one nanopore.

18. The method of claim 17, including applying a voltage difference to a first electrode pair in the at least one nanopore.

19. The method of claim 18, including applying voltage differences to a plurality of different electrode pairs in the at least one nanopore.

20. The method of claim 19, including (a) simultaneously electrically probing the chemical substance with the plurality of different electrode pairs in the at least one nanopore as the chemical substance passes through the at least one nanopore and (b) influencing flow of the chemical substance through the at least one nanopore through application of dielectrophoretic forces.
